(19) United States
(12) Patent Application Publication
(10) Pub. No.: US 2022/0137021 A1
(43) Pub. Date: May 5, 2022

(54) System and Method for Automatically Monitoring and Tracking the Chemistry of a Managed Body of Water

(71) Applicant: Brian Zima, Upper Saddle River, NJ (US)

(72) Inventor: Brian Zima, Upper Saddle River, NJ (US)

(21) Appl. No.: 17/515,317

(22) Filed: Oct. 29, 2021

Related U.S. Application Data
(60) Provisional application No. 63/108,252, filed on Oct. 30, 2020.

(51) Int. Cl. G01N 33/18; G01N 21/78

(52) U.S. Cl. CPC G01N 33/18; G01N 21/78

(57) ABSTRACT

A test system and method for determining the concentrations of conditioning chemicals in a body of managed water. A test unit is provided. The test unit has a test chamber, a supply of reagents, an array of LEDs, an optical sensor, and at least one diaphragm valve assembly. The diaphragm valve assembly is used to supply precise volumes of reagents into the test chamber. The reagents are mixed with a water sample to create a test mixture. The reagents react with the conditioning chemicals and provides a color profile. The array of LEDs shines colored light through the test mixture. The test mixture absorbs the light that corresponds to the color profile of the test mixture. The remaining light passes through the test mixture and is detected by optical sensors. The optical sensors generate data that is indicative of the concentrations of the conditioning chemicals.

### RELATED APPLICATIONS

This application claims priority of U.S. Provisional Application No. 63/108,252, filed Oct. 30, 2020.

### BACKGROUND OF THE INVENTION

### 1. Field Of The Invention

In general, the present invention relates to systems and methods that are used to test the chemistry of a managed body of water to ensure that the water is safe for people.

### 2. Prior Art Description

Many state and local municipalities have standards regarding the chemistry of water in swimming pools, large fountains, water amusements, water treatment plants and other managed bodies of water s in which water must be actively filtered and treated. The standards typically provide acceptable ranges for water pH, chlorine content, alkalinity, calcium hardness, cyanuric acid, and dissolved solids. These variables must be monitored and controlled by the administrators of the managed body of water.

For most administrators for bodies of water, the chemistry of the water is monitored by periodically taking small samples of water and mixing those samples with various chemical reagents. Concentrations of various chemicals are then determined by observing the changes in the reagents as they react with the sampled water.

This testing process has many problems. Primary among the problems is human error. Testing water in a managed body of water is traditionally a two-step process. Both steps are highly susceptible to human error. In the first step, water samples are collected and are mixed with reagents. Water samples may not collected at the exact times they are scheduled. As such, the period from the last water treatment varies for each sample. The samples are typically manually drawn from a pool. As such, the volume of the water drawn for the sample is likely to vary between samples. The reagents are manually added to the samples. As such, the volume of the reagents can vary also between tests. The volume of water samples collected, the temperature of the water samples, and/or the ratio of water to reagents all vary from test to test. These variables affect the results of a test and cause inconsistent results to be gathered.

In the second step of the testing process, the sample of water is mixed with reagents and the reagents change the color of the collected samples. The results of the tests are then quantified using visual color comparisons. That is, the color of the collected samples are visually compared to a color chart in order to determine a numerical value for the concentration of chemicals and/or contaminants present. It will be understood that over eight percent of the male population is color blind to some degree. In addition, a percentage of the population has some degree of visual impairment that effects the ability to accurately compare colors. Further, if the water of a pool is being tested by a worker at the pool, it is not uncommon for that worker to be wearing sunglasses or tinted glasses. Sunglasses, tinted glasses and polarized eyewear significantly decrease the ability of a person to distinguish between shades of color. As a consequence, the perceived results of a water test can vary greatly from person to person and from day to day. This makes the water tests inaccurate, therein causing too much, too little, or incorrect additives to be added to the water.

In the prior art, attempts have been made to improve water testing results by removing some of the human factors. In some prior art systems, the chemistry of water is tested using electrodes. Such prior art systems are exemplified by U.S. Patent Application Publication No. 2009/0057145 to Vincent and U.S. Patent Application Publication No. 2017/0248568 to Yizhack. However, there are problems associated with using electrodes. One problem is that test results vary depending upon the pH of the water and the concentration of electrolytes in the water. Some pools are filled with well water that contains a high concentration of dissolved minerals. Salt is also often added to pools to soften the water or to intentionally create brine. These electrolytes make the water more conductive and greatly affect the measured results of electrode sensors.

Some prior art systems try to eliminate human error by replacing human eyes with automated camera systems. The camera systems are used to determine the exact color of a water sample that has been mixed with a reagent. Such prior art systems are exemplified by U.S. Pat. No. 9,651,534 to Ehlert. Although such systems eliminate errors caused by color blindness and other color detection deficiencies, such testing equipment does nothing to eliminate errors caused during the collection of the samples and the mixing of samples with reagents. Furthermore, camera systems can be readily stymied by the presence of colorants and/or particulate matter within the collected sample. For instance, if a camera system is viewing water that is dirty or water that is colored with iron or other minerals, the color of the reagent in the water is likely to be perceived by the camera as darker than it actually is. In such an instance, the camera system may generate data that does not actually represent the chemistry of the water being tested.

A need therefore exists for a water chemistry sampling system that eliminates human error from the collected data and generates accurate results regardless of water quality. This need is met by the present invention as described below.

### SUMMARY OF THE INVENTION

The present invention is a test system and method for determining the concentrations of conditioning chemicals in a body of managed water that is actively filtered. A test unit is provided. The test unit can be affixed to the filtration system for the body of water or can be portable. If affixed to the filtration system, testing can be automated, therein removing many errors traditionally caused by human error. The test unit has a test chamber, a supply of reagents, an array of LEDs, an optical sensor, and at least one calibrated diaphragm valve assembly. The diaphragm valve assembly supplies a controlled volume of one or more reagents into the test chamber. The reagents are mixed with a water sample that is drawn from the body of water. The reagents and the water sample create a test mixture. The reagents react with the conditioning chemicals in the water sample. This causes the test mixture to change color. The test mixture, therefore, creates a color profile that is indicative of the concentration of conditioning chemicals present.

The array of LEDs is used to shine colored light through the test mixture. The LEDs have different color frequencies and create an overall color light profile. As the LEDs shine through the test mixture, the test mixture absorbs the light that corresponds to the color profile of the test mixture. The remaining light passes through the test mixture and is detected by one or more optical sensors. The optical sensors generate data that is indicative of the concentrations of the conditioning chemicals present in the water sample. The data is converted into a readable format for display on a screen or a smart phone.

### BRIEF DESCRIPTION OF THE DRAWINGS

For a better understanding of the present invention, reference is made to the following description of exemplary embodiments thereof, considered in conjunction with the accompanying drawings, in which:

FIG. 1 is a schematic showing a managed body of water, in the form of a pool, in conjunction with a first exemplary embodiment of the present invention automated testing system;

FIG. 2 is a schematic of the first exemplary embodiment of the automated testing system;

FIG. 3 is a schematic of a pneumatically controlled diaphragm valve assembly in an intake configuration;

FIG. 4 is a schematic of a pneumatically controlled diaphragm valve assembly in an output configuration;

FIG. 5 is a graph showing an exemplary color profile for a test mixture;

FIG. 6 is a graph showing a full color range for an array of LEDs;

FIG. 7 is a representation showing the full color range of FIG. 4 becoming diminished by the color profile of FIG. 3 by passing through a test sample;

FIG. 8 shows result data collected using the automated testing system; and

FIG. 9 shows a schematic of a second exemplary exemplary embodiment of the automated testing system.

### DETAILED DESCRIPTION OF THE DRAWINGS

Although the present invention test system can be embodied in many ways, only two exemplary embodiments are illustrated. The selected embodiments show a fixed testing system and a mobile testing system. The exemplary embodiments are being shown for the purposes of explanation and description. The exemplary embodiments are selected in order to set forth two of the best modes contemplated for the invention. The illustrated embodiments, however, are merely exemplary and should not be considered limitations when interpreting the scope of the claims.

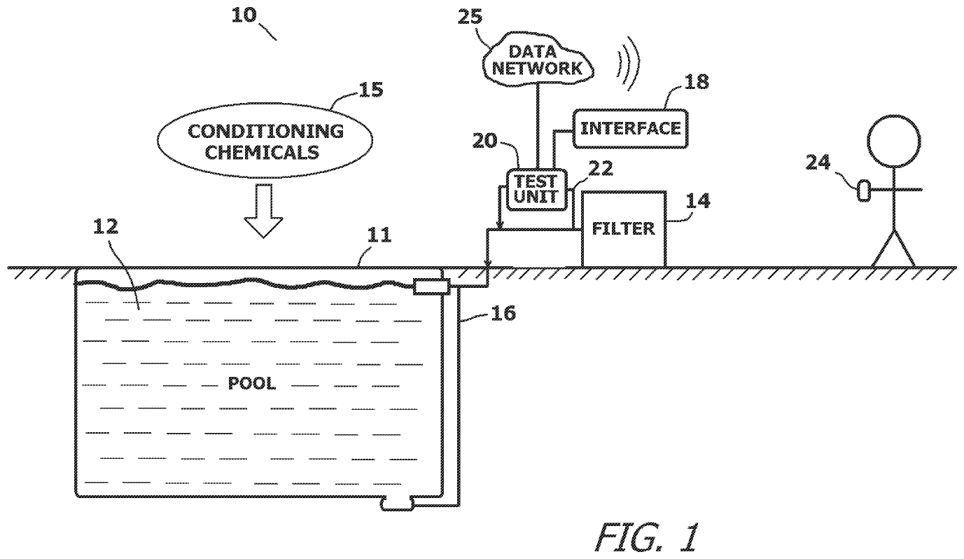

Referring to FIG. 1, a managed body of water 10 is shown in a form of a pool 11. The pool 11 contains a volume of pool water 12. It will be understood that the illustrated pool 11 is exemplary of any managed body of water, such as a fountain, water-based amusement park ride, pedestrian cooling station, or the like that uses water that is is maintained with conditioning chemicals 15. The present invention is a system and method for automatically testing the water 12 to obtain the concentrations of the conditioning chemicals 15 and recording the results for later review or action. The pool 11 has a filter system 14 that is used to remove contaminants from the water 12.

The filter system 14 recycles the water 12 from the pool 11 through various filter lines 16.

A testing unit 20 is provided. The testing unit 20 is connected to the filter system 14 via a tap line 22. The tap line 22 receives a supply of the water 12 from one or more of the filter lines 16 used by the filter system 14. It is preferred that the water 12 used for sampling has already passed through the filter system 14. As will be explained, the testing unit 20 chemically tests the pool water 12 in an automated fashion that eliminates many errors that can occur using traditional manual testing methods. The testing unit 20 saves the results of each test. The test results can be accessed at the testing unit 20 using an interface 18. Alternatively, the testing unit 20 can forward the results to a remote computing device 24, such as a smart phone, through a data network 25. In this manner, the quality of water 12 can be monitored remotely by the pool administrator or a municipal health official. Furthermore, different test results can be compared over time to detect trends and the possible need for pool maintenance.

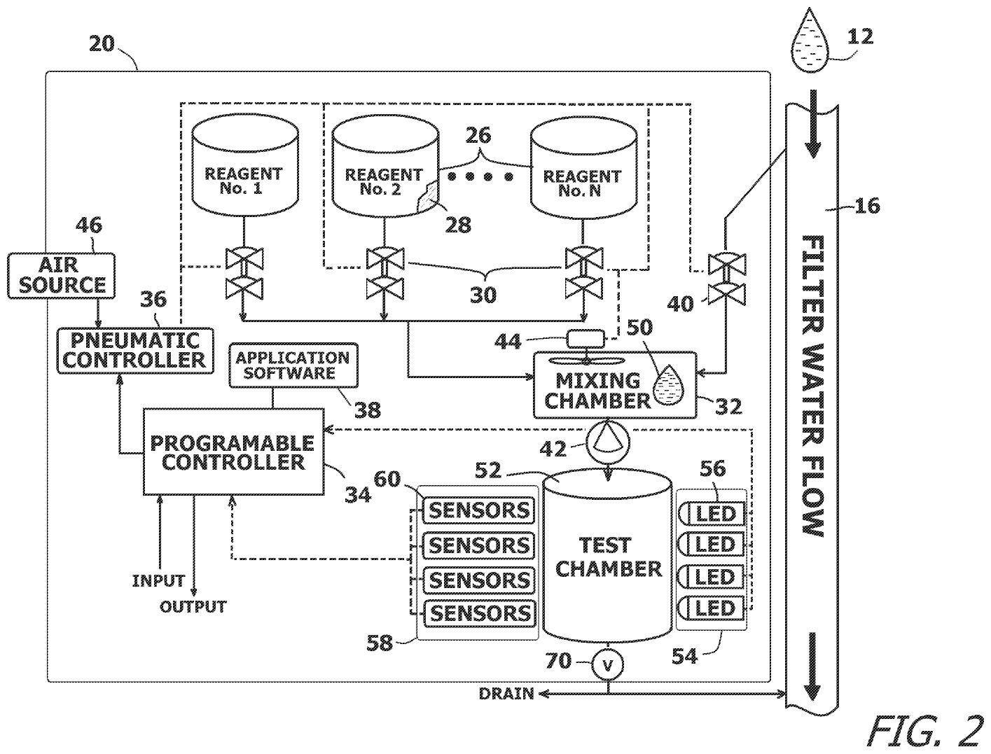

Referring to FIG. 2 in conjunction with FIG. 1, it can be seen that the testing unit 20 contains a plurality of reagent reservoirs 26. The reagent reservoirs 26 contain traditional reagents 28 for testing water. Such reagents include, but are not limited to, N,N diethyl-1, 4 phenylenediamine sulfate, 7-hydroxyphenoxazone chromophore, orthotolidine, pH equalizers, and/or chemical equivalents to These reagents. With such reagents 28, traditional water tests, such as a DPD tests, TC tests, CC tests, and pH tests can be performed. DPD tests measure oxidizer levels in the water 12. TC tests measure the total chlorine in the water 12. CC tests measure the combined chlorine in the water 12. Lastly, pH tests measure the acidity and/or alkalinity of the water 12.

The reagent reservoirs 26 are sealed and hold the reagents 28 in volumes sufficient to perform dozens of tests. The preferred volume of the reagents 28 is sufficient to supply enough reagents 28 to perform between at least one week and one month of water testing. In this manner, the reagent reservoirs 26 do not require refilling or replacing every day. However, they are filled or replaced often enough to remain fresh and chemically active.

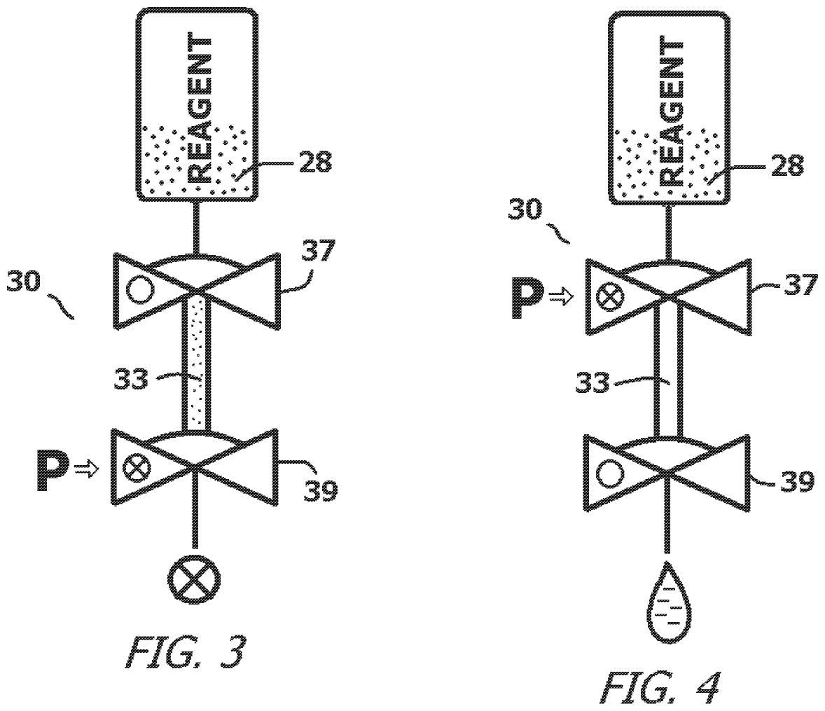

A calibrated diaphragm valve assembly 30 is provided for each of the reagent reservoirs 26. The diaphragm valve assemblies 30 are pneumatically powered. Referring briefly to FIG. 3 and FIG. 4, it can be seen that each diaphragm valve assembly 30 contains a calibrated chamber 33 that holds a precise volume of a reagent 28. The calibrated chamber 33 is interposed between two diaphragm valves, which include an intake diaphragm valve 37 and an output diaphragm valve 39. Prior to a test being performed, the intake diaphragm valve 37 opens and the calibrated chamber 33 fills with a precise volume of a reagent 28. See FIG. 3. The volume selected for the calibrated chamber 33 depends upon the reagent 28 and the test being performed. When a test is performed, the intake diaphragm valve 33 closes and the output diaphragm valve 39 opens. See FIG. 4. The volume of the reagent 28 that was in the calibrated chamber 33 now is released for the test. In this manner, by simply applying pneumatic pressure, a precise volume of a reagent 28 can be drawn out of a reagent reservoir 26 for use in testing. Each diaphragm valve assembly 30 should have an accuracy of at least 0.05 milliliters per cycle.

The reagents 28 moved by the diaphragm valve assemblies 30 are displaced into an optional mixing chamber 32 and then into a test chamber. Returning to FIG. 2, a mixing chamber 32 is shown. In the mixing chamber 32, the reagents 28 are mixed with water prior to being forwarded to a test chamber 52. It will be understood that the use of the mixing chamber 32 is optional and that reagents 28 and water can be released directly into the testing chamber 52 if sufficient natural mixing occurs.

The operation of the diaphragm valve assemblies 30 is regulated by a pneumatic controller 36. The pneumatic controller 36 is operated by a programable controller 34. The programable controller 34 runs application software 38. The pneumatic controller 36 powers the diaphragm valve assemblies 30 and other mechanical components, such as a tap diaphragm valve assembly 40, a transfer diaphragm valve 42, and an agitator 44. A source of compressed air 46 is coupled to the pneumatic controller 36 and provides the compressed air needed to operate the various pneumatic components. The diaphragm valve assemblies 30 are coupled to the reagent reservoirs 26 can be operated with precision to add precise volumes of the reagents 28 into the mixing chamber 32 at any programmed time.

The tap diaphragm valve assembly 40 is used to add a specific amount of water 12 to the mixing chamber 32. The water 12 is obtained from the tap line 22.

If a mixing chamber 32 is used, an agitator 44 can be provided to agitate the water 12 and reagents 28 within the mixing chamber 32. This creates a test mixture 50. The volume of the water 12 and the volume of the reagents 28 are precisely controlled. Accordingly, the test mixture 50 is precise and contains no human measurement errors. Furthermore, the time that the sample of water 12 is drawn is precisely controlled by the cycle capacity of the tap diaphragm valve assembly 40. Accordingly, the same test can be performed daily at the same times, therein removing other potential testing errors.

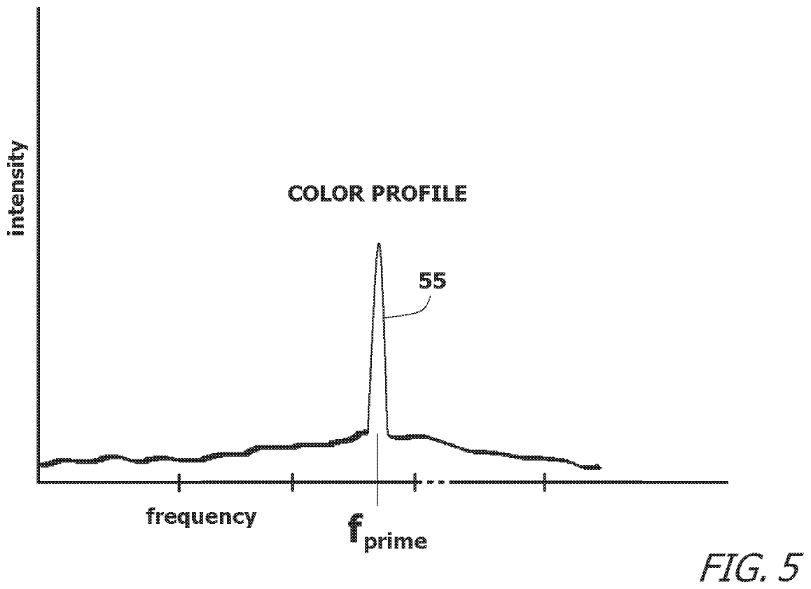

Referring to FIG. 2 in conjunction with FIG. 5, it can be seen that in the mixing chamber 32, the agitator 44 stirs the test mixture 50 until the reagents 28 have time to fully react with the water 12. As the reagents 28 react with the pool water 12, the test mixture 50 adopts a certain color profile 55 (FIG. 5) with an associated pellucidity. The transfer pneumatic valve 42 is opened and the tinted test mixture 50 is advanced into a test chamber 52. The test chamber 52 can be transparent or can contain transparent windows. An array 54 of LEDs 56 is provided. The LEDs 56 are oriented to shine light through the test chamber 52 and the test mixture 50 contained within the test chamber 52. One or more optical sensors 60 are provided. In the shown embodiment, an array 58 of optical sensors 60 is provided. The optical sensors 60 are positioned across from the LEDs 56 so as to receive the light shining from the LEDs 56 through the test mixture 50.

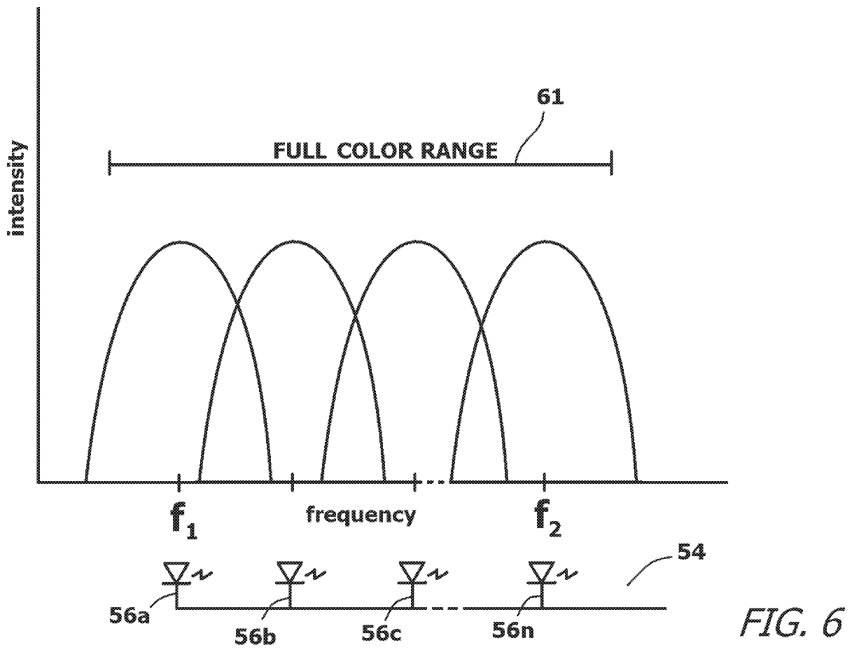

The individual LEDs 56 are preferably monochromatic or near monochromatic. However, different. LEDs 56 emit light at different color frequencies. Different LEDs 56 in the array 54 are used to perform different tests. Referring to FIG. 6 in conjunction with FIG. 2 and FIG. 5, the relationship between a test and the LEDs 56 is explained. In certain tests, the color profile of the test mixture 50 will vary between a light color and a dark color, depending upon the concentration of conditioning chemicals in the test mixture 50. The light color has a first primary frequency (f₁) that corresponds to its primary color. The dark color has a second primary frequency (f₂) that correspond to its primary color. As such, it will be understood that the results of a particular water test will fall within the boundaries of a full color range 61. Where the color profile 55 (FIG. 5) of a test mixture 50 falls within with the full color range 61 is indicative of the concentration of a particular conditioning chemical or contaminant in the water 12 being tested. If the color profile 55 of the test mixture 50 falls to either end of the full color range 61, too much or too little of a conditioning chemical 15 may be present. In many tests, if the color profile 55 of the test mixture 50 falls near the center of the full color range 61, then the water 12 contains the proper amount of conditioning chemicals 15.

The LEDs 56 utilized in the test chamber 52 are each colored to cover the full color range of the test. A first LED 56a shines light at a frequency near the first primary frequency (f₁) in the possible color range. A last LED 56n shines light at a frequency near the second primary frequency (f₂). At least one interim LED 56b, 56c shines light at a frequency between the first primary frequency (f₁) and the second primary frequency (f₂). The result is the full LED color range 61 shown in FIG. 6.

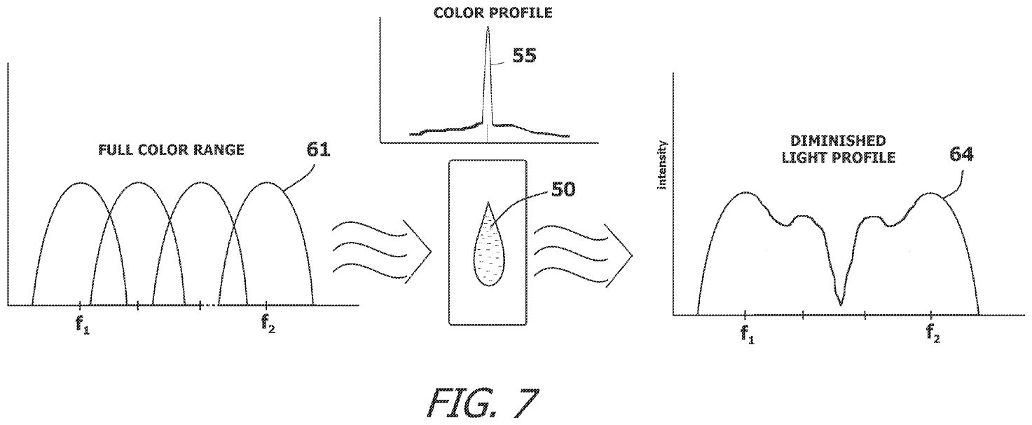

The array 54 of LEDs 56 produces the full color range 61 shown in FIG. 6. This full color range 61 shines through the test chamber 52. The color profile 55 of the test mixture 50 corresponds to only some of the light frequencies contained in the full color range 61. Referring to FIG. 7 in conjunction with FIG. 2, FIG. 5 and FIG. 6, it will be understood that when the full color range 61 of the LED array 54 shines through the test mixture 50, some of the light is absorbed by the color profile 55 of the test mixture 50. The frequencies of light absorbed correspond to the frequencies contained within the color profile 55 of the test mixture 50. As a result, the full color range 61 is diminished in the frequencies that correspond to the color profile 55 of the test mixture 50. This produces a diminished light profile 64. The diminished light profile 64 is detected by the optical sensors 60 in the optical array 58. The data produced by the optical array 58 is analyzed by the programmable controller 34. The programmable controller 34, and the application software 38 it runs, can precisely determine the color profile 55 and/or the pellucidity of the test mixture 50. The application. software 38 compares the data from the diminished profile 64 to a reference database, wherein concentration levels for the conditioning chemicals are substituted for the color frequencies in the diminished profile 64.

After the test, the mixing chamber 32 and the test. chamber 52 are cleaned by flushing the mixing chamber 32 and the test chamber 52 with the water 12 from the filter system 14. A flush valve 70 is opened to drain the test chamber 52. The tap diaphragm valve assembly 40 is cycled. to enable water to flood the mixing chamber 32 and test chamber 52. The flush of new water is continued until the mixing chamber 32 and the test. chamber 52 are clean. The tap diaphragm valve assembly 40 is then closed and both the mixing chamber 32 and test chamber 52 are allowed to drain. The test unit 20 is then ready for reuse.

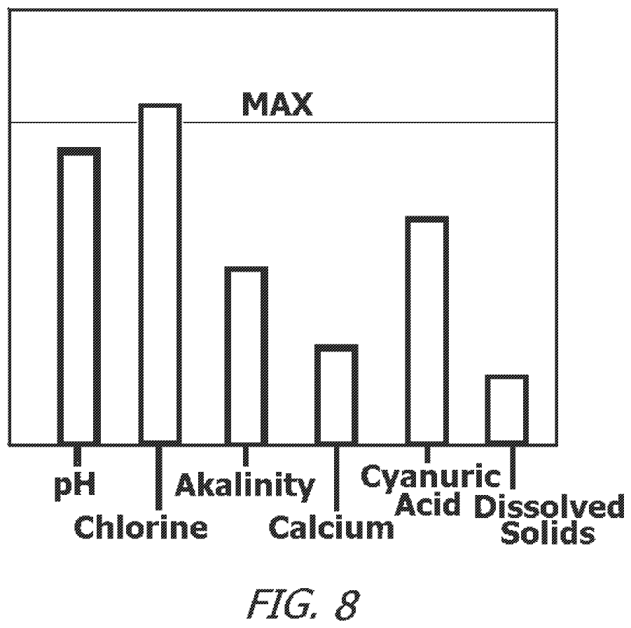

Referring to FIG. 8 in conjunction with FIG. 2, it can be seen that using the application software 38, the programmable controller 34 can. cause various reagents 28 to react with the water 12 in battery of tests. The test produces data on. the chemistry of the water 12, such as pH, chlorine content, alkalinity, calcium hardness, cyanuric acid, and dissolved solids. The application software 38 processes the test data into useful display information that can be viewed by the administrator of the body of water. The administrator can use the collected data to automatically operate filters, chlorine injectors, and other chemical injectors, therein maintaining a controlled water chemistry. The data can also be stored and supplied in a report for a pool inspector or other compliance authority.

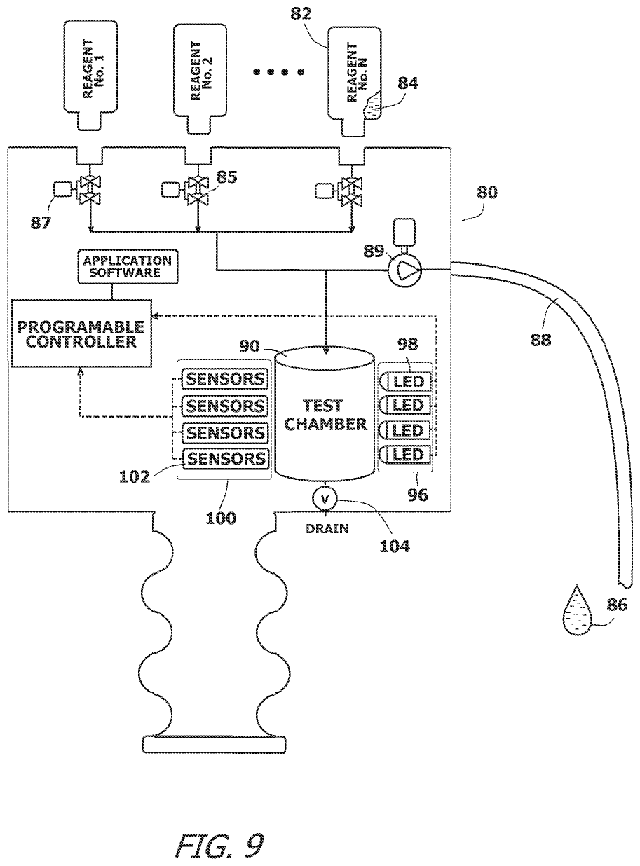

Referring to FIG. 9, a handheld version of a testing unit 80 is shown. The handheld testing unit 80 is useful to health inspectors and other officials that are responsible for testing the quality of water in a variety of different waterworks. In this version, reagents 84 are attached to the testing unit 80 in removable cartridges 82. Diaphragm valve assemblies 85 are provided that are powered by electrical solenoids 87. The diaphragm valve assemblies 85 are used to move precise volumes of a reagent 84 into a test chamber 90. Water 86 is drawn into the testing unit 80 using a sampling tube 88 and a tap diaphragm pump 89. The tap diaphragm pump 89 injects a precise volume of the water 86 into the test chamber 90. The overall testing unit 80 can then be manually shaken to produce a test sample. As such, the need for a separate mixing chamber and/or agitator is removed.

The test chamber 90 is transparent and is interposed between an array 96 of LEDs 98 and an array 100 of optical sensors 102. The LEDs 98 and optical sensors 102 evaluate the test sample 94 in the same manner as has previously been described. Once the test is complete and the data collected, a drain valve 104 is opened and the test chamber 92 is drained.

It will be understood that the embodiments of the present invention that are illustrated and described are merely exemplary and that a person skilled in the art can make many variations to those embodiments. All such embodiments are intended to be included within the scope of the present invention as defined by the appended claims.

What is claimed is:

1. In a body of water that is actively filtered by a filter system and is treated with conditioning chemicals, a method of performing a test that quantifies concentrations for at least some of said conditioning chemicals, said method comprising:
providing a test unit that contains a test chamber, a supply of reagents, an array of LEDs, an optical sensor, and at least one valve for selectively releasing a controlled volume of at least one of said reagents into said test chamber;
drawing a water sample from said body of water into said test chamber, wherein said water sample mixes with any of said reagents in said test chamber, therein forming a test mixture;
wherein said reagents react with at least one of said conditioning chemicals in said water sample to provide said test mixture with a color profile, wherein said color profile is indicative of said concentrations of said at least one of said conditioning chemicals present in said water sample;
shining light produced by said array of LEDs through said test mixture in said test chamber, wherein said color profile of said test mixture absorbs some of said light,
detecting said light that passes through said test mixture with said optical sensor, wherein said optical sensor creates data indicative of said concentrations said at least one of said conditioning chemicals present in said water sample; and
converting said data into a readable display.

2. The method according to claim 1, wherein said at least one valve for selectively releasing said controlled volume of at least one of said reagents into said test chamber is a diaphragm valve.

3. The method according to claim 2, wherein said diaphragm valve is pneumatically controlled.

4. The method according to claim 1, wherein said array of LEDs contains LEDs of different colors, wherein said array of LEDs produces light in a color range.

5. The method according to claim 2, wherein said color profile of said test mixture is contained within said color range produced by said array of LEDs.

6. The method according to claim 1, wherein said test unit is hydraulically attached to said filter system with a tap line and drawing said water sample includes transferring said water to said test unit from said filter system through said tap line.

7. The method according to claim 1, further including flushing said test chamber with said water after detecting said light that passes through said test mixture with said optical sensor.

8. The method according to claim 2, wherein test unit includes a pneumatic controller for controlling pneumatic pressure suppled to said diaphragm valve.

9. The method according to claim 1, wherein said test unit further includes a mixing chamber, wherein said controlled volume of at least one of said reagents is mixed with said water sample to produce said test mixture prior to said test mixture being directed to said test chamber.

10. The method according to claim 1, wherein said test mixture is actively mixed prior to shining light produced by said array of LEDs through said test mixture.

11. A method of performing a test that quantifies concentrations of conditioning chemicals water of a pool, said method comprising:
providing a test unit that contains a test chamber, a supply of reagents, an array of LEDs, an optical sensor, and diaphragm valves;
drawing a water sample from said water into said test chamber,
pneumatically operating said diaphragm valves to release a controlled volume of at least one of said reagents into said test chamber, wherein said water sample mixes with said at least one of said reagents, therein forming a test mixture, wherein said reagents react with at least one of said conditioning chemicals in said water sample to provide said test mixture with a color profile;
shining light produced by said array of LEDs through said test mixture in said test chamber, wherein said color profile of said test mixture absorbs some of said light,
detecting said light that passes through said test mixture with said optical sensor, wherein said optical sensor creates data indicative of said concentrations of said at least one of said conditioning chemicals present in said water sample.

12. The method according to claim 11, wherein said array of LEDs produces light in a color range.

13. The method according to claim 12, wherein said color profile of said test mixture is contained within said color range produced by said array of LEDs.

14. The method according to claim 11, wherein said test unit automatically draws said water sample and tests said water sample at preprogrammed times.

15. The method according to claim 11, further including flushing said test chamber with said water after detecting said light that passes through said test mixture with said optical sensor.

16. The method according to claim 11, wherein said test unit further includes a mixing chamber, wherein said at least one of said reagents is mixed with said water sample in said mixing chamber to produce said test mixture prior to said test mixture being directed to said test chamber.

17. The method according to claim 11, wherein said test mixture is actively mixed prior to shining light produced by said array of LEDs through said test mixture.

18. A testing unit that quantifies concentrations for at least some of said conditioning chemicals present in water, comprising:
a test chamber for holding a sample of said water;
a supply of reagents;
at least one valve assembly for releasing a controlled volume of at least one of said reagents into said test chamber to create a test mixture with said water within said test chamber;
an array of LEDs that shine light through said test mixture; and
at least one optical sensor for detecting frequencies of light passing through said test mixture from said array of LEDs.

19. The testing unit according to claim 18, wherein said at least one valve assembly includes a set of pneumatically powered diaphragm valves disposed at opposite ends of a calibrated chamber.

20. The testing unit according to claim 18, wherein said array of LEDs contains LEDs of different colors.
